(19) United States
(12) Patent Application Publication
(10) Pub. No.: US 2007/0051626 A1
(43) Pub. Date: Mar. 8, 2007

(54) Cataphoresis apparatus cataphoresis method, and detection method for organism-related material using the apparatus and the method

(76) Inventors: Chiho Itou, Otake-shi (JP); Tetsuya Jigami, Yokohama-shi (JP); Teruta Ishimaru, Yokohama-shi (JP); Noriyuki Ogawa, Toyama-shi (JP); Masakazu Minagawa, Toyohashi-shi (JP); Yasuo Hiromoto, Otake-shi (JP); Toshinori Sumi, Otake-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/573,158

(22) PCT Filed: Sep. 27, 2004
(86) PCT No.: PCT/JP04/14097

§ 371 Date: Nov. 13, 2006

(30) Foreign Application Priority Data
Sep. 26, 2003 (JP) 2003-335782

(51) Int. Cl. C07K 1/26; G01N 27/00

(52) U.S. Cl. 204/456; 204/606

(57) ABSTRACT

An electrophoresis apparatus that has a gel retaining layer; one or two sample solution storage portions disposed on either side or both sides of said gel retaning layer; two semi-permeable membranes disposed on the outer sides of said sample solution storage portions; buffer solution storage portions disposed on the outer sides of said semi-permeable membranes; a pair of electrodes disposed on the outer sides of said buffer solution storage portions; and at least one liquid inlet/outlet respectively provided in each of the sample solution storage portions and the buffer solution storage portions.

### TECHNICAL FIELD

The present invention relates to an electrophoresis apparatus and an electrophoresis method suited to detection of biological material such as nucleic acid.

### BACKGROUND ART

In recent years, biochips known as DNA microarrays and DNA chips have been developed for disease diagnosis and investigation. Methods of manufacturing biochips include a method of direct solid phase synthesis of a short-chain nucleic acid using photolithographic techniques on a substrate of silicon and the like (Patent Documents 1 and 2) and a method of immobilizing biological probes (hereinafter referred to Simply as “probes”) such as nucleic acids onto a chemically or physically modified substrate by spotting method (Non-Patent Document 1). There is also known an immobilization method that involves binding a plurality of hollow fibers in resin, introducing a probe-immobilized gel in the hollow portion of each hollow fiber, and then obtaining a section thereof by slicing in a direction perpendicular to the gel-holding hollow fiber (Patent Document 3). Methods such as that disclosed in Patent Document 3 can retain a probe not only on the surface of a chip but in the thickness direction as well. This allows the introduction of a large volume of probes to obtain a high detection sensitivity.

In addition, electrophoresis can increase the hybridization efficiency of biochips in which such probes are immobilized in gel. Hybridization methods employing electrophoresis include a method that performs a hybridization reaction and washing away of excess samples that are not hybridized at a high-speed (Patent Document 4).
Non-Patent Document 1: Science, Oct. 1995, Vol. 270, No. 5235, 467-470
Patent Document 1: U.S. Pat. No. 5,445,934 specification
Patent Document 2: U.S. Pat. No. 5,774,305 specification
Patent Document 3: Japanese Unexamined Patent Application, First Publication No. 2000-270878
Patent Document 4: Japanese Unexamined Patent Application, First Publication No. 2000-60554

### DISCLOSURE OF THE INVENTION

Problems to be Solved by the Invention

However, in the method disclosed in Patent Document 4, the sample solution and the electrodes are in contact. As a result, the hybridization efficiency falls due to adsorption of the sample molecules onto the electrodes and decomposition of the sample molecules due to the electrode reaction caused by electrolysis.

Also, since the sample solution and the electrodes are in contact, bubbling of gas generated at the electrodes due to electrolysis prevents migration of the sample molecules in the intended direction, and thus additional time is required for the hybridization reaction and washing treatment.

An object of the present invention is to provide an electrophoresis apparatus and an electrophoresis method that can suppress adsorption of sample molecules onto the electrodes and decomposition of the sample molecules due to the electrode reaction caused by electrolysis and eliminate the effects of gas generated from the electrodes by the electrolysis in order to attain a high hybridization efficiency.

Another object of the present invention is to provide an electrophoresis apparatus and electrophoresis method that enable washing treatment in a short time.

Means for Solving the Problem

The present invention is an electrophoresis apparatus that has a gel retaining layer; one or two sample solution storage portions disposed on either side or both sides of said gel retaining layer, two semi-permeable membranes disposed on the outer sides of said sample solution storage portions; buffer solution storage portions disposed on the outer sides of said semi-permeable membranes; a pair of electrodes disposed on the outer sides of said buffer solution storage portions; and at least one liquid inlet/outlet respectively provided in each of the sample solution storage portions and the buffer solution storage portions.

The present invention also is an electrophoresis method comprising the steps of: feeding a sample solution to sample solution storage portions and applying a voltage across the pair of electrodes while feeding a buffer solution to a buffer solution storage portions, using said electrophoresis apparatus.

Advantageous Effects of the Invention

Since the electrophoresis apparatus and the electrophoresis method of the present invention can restrict adsorption of sample molecules onto the electrodes and decomposition of the sample molecules due to the electrode reaction caused by electrolysis and can eliminate the influence of gas by quickly discharging the gas that is generated from the electrodes by the electrolysis, the hybridization efficiency can be increased. The present invention can also perform washing treatment in a short time.

### BRIEF DESCRIPTION OF THE DRAWINGS

FIG. 1 is a schematic diagram showing an example of the electrophoresis apparatus according to the present invention in which the electrophoresis portion 100, which is surrounded by a dashed line, is in shown in an exploded view.

FIG. 2 is an exploded view of the electrophoresis portion of FIG. 1.

FIG. 3 is a cross-sectional view of the electrophoresis portion 100 along line A-A′ in FIG. 2.

FIG. 4 is a conceptual view showing another example of the electrophoesis apparatus of the present invention; in this example, there is one sample solution storage portion.

FIG. 5 is a schematic drawing showing the electrophoresis portion of FIG. 4.

FIG. 6 is a cross-sectional view of the electrophoresis portion 102 along line B-B′ in FIG. 5.

FIG. 7 is a cross-sectional view showing the electrophoresis portion of comparative example 2.

FIG. 8 is a cross-sectional view along line C-C′ in FIG. 3.

### DESCRIPTION OF THE REFERENCE SYMBOLS

1 electrode
2 buffer solution storage portion
3 semi-permeable membrane
4 sample solution storage portion
5 gel retaining layer
6 sample solution storage portion
7 semi-permeable membrane
8 buffer solution storage portion
9 electrode
22 carrier gel
32 sample solution container
42 temperature controller
43 arbitrary waveform generator
44 coordination control device

### BEST MODE FOR CARRYING OUT THE INVENTION

Embodiments for carrying out the present invention will be described below, with reference to the attached drawings.

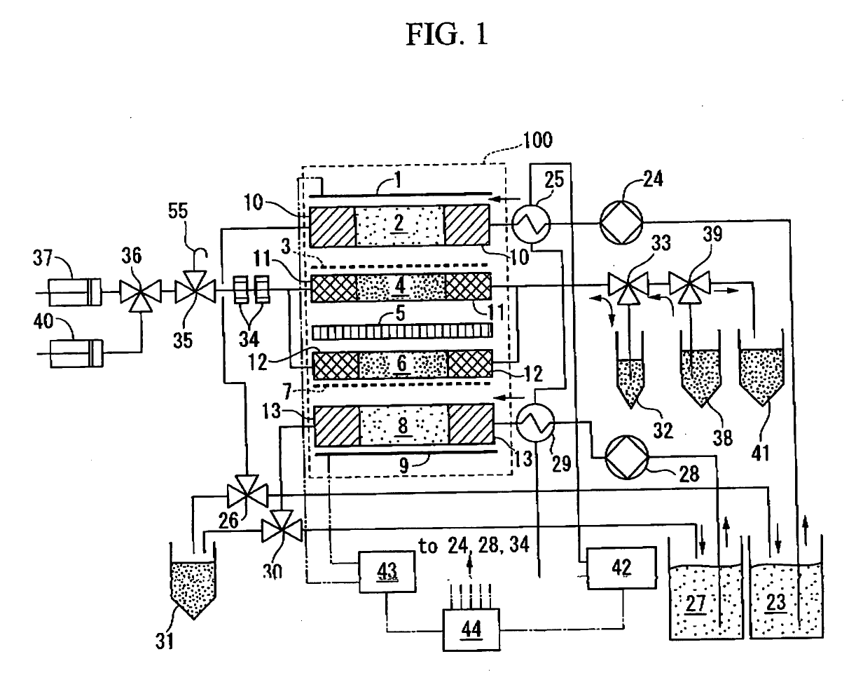

FIG. 1 is a schematic diagram showing a first embodiment of the electraphoresis apparatus of the present invention, having a structure in which sample solution storage portions are disposed on both outer sides of a gel retaining layer. An electropboresis portion 100 of the electrophoresis apparatus is shown as an exploded view. The electrophoresis portion 100 comprises a gel retaining layer 5, two sample solution storage portions 4 and 6 disposed on the outer sides thereof, two semi-permeable membranes 3 and 7 disposed on the outer sides thereof, buffer solution storage portions 2 and 8 disposed on the outer sides thereof, and a pair of electrodes 1 and 9 disposed on the outer sides thereof.

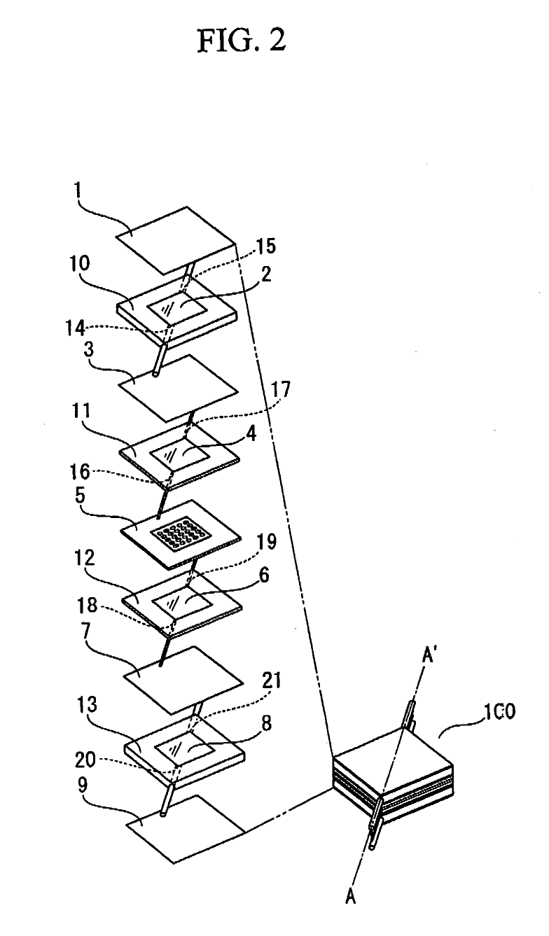

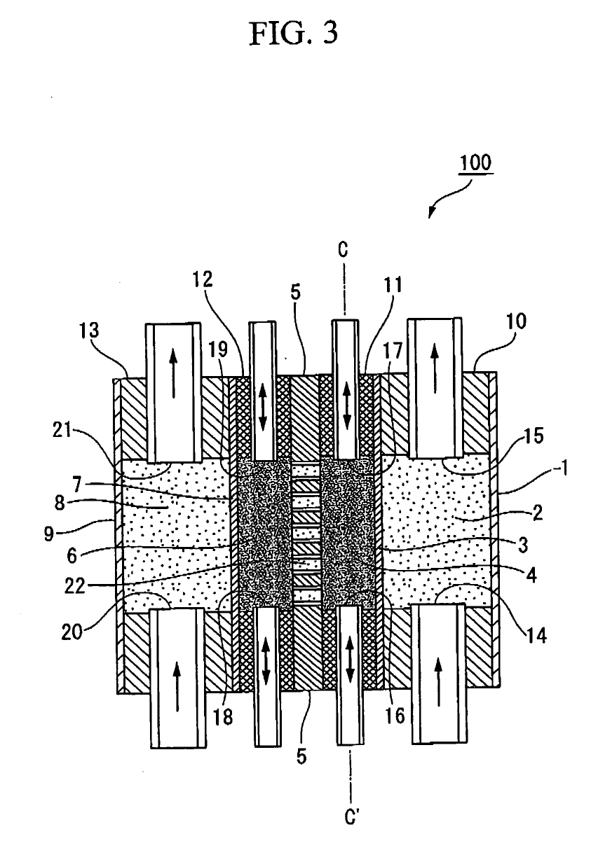

FIG. 2 is a perspective view of the electophoresis portion 100 and a perspective view of the component members thereof. FIG. 3 is a sectional view showing the cross section along line A-A′ of the electrophoresis portion in FIG. 2. In both FIG. 1 and FIG. 2, the stacking surfaces of the component members of the electrophoresis portion are illustrated as being stacked in a horizontal direction. However, the stacking surfaces of the component members are actually arranged in a stack in the vertical direction as shown in FIG. 3.

In this example, the sample solution spacers 11 and 12 are arranged on both outer sides of the gel retaining layer 5, with the semi-permeable membranes 3 and 7 being disposed on the outer sides thereof. Each of the sample solution spacers 11 and 12 has a hollow portion passing from the surface contacting the gel retaining layer 5 to the surface on the opposite side, with the hollow portion forming the sample solution storage portion 4 and 6, respectively. One surface of the sample solution storage portion 4 and 6 contacts the gel retaining layer 5, while another surface thereof contacts the semi-permeable membranes 3 and 7, respectively.

The buffer solution spacers 10 and 13 are arranged on the outer side of the semi-permeable membranes 3 and 7, respectively. The buffer solution spacers 10 and 13 have a hollow portion passing from the surface contacting the semi-permeable membranes 3 and 7, respectively, to the surface on the opposite side, with the hollow portion respectively forming the buffer solution storage portions 2 and 8, respectively. One surface of the buffer solution storage portions 2 and 8 respectively contacts the semi-permeable membranes 3 and 7, while the other surface is sealed by the electrodes 1 and 9, respectively. Voltage is applied on the gel retaining layer 5 from the outside using the electrodes 1 and 9.

The gel retaining layer 5 has a constitution in which the sample solution contained in the adjacent sample solution storage portions 4 and 6 can come into contact with a gel. It has, for example, a constitution that retains gel in a through-hole portion of a porous plate in which one or more through-holes are provided. FIG. 3 shows the gel retaining layer in which gel material is held in a plurality of through-holes formed at a specified interval in a plate. Publicly known gel, such as acrylamide-based gel and agarose gel, can be used as the gel material. Biological materials, such as DNA probes, are bound to the gel material. When performing electrophoresis, biological specimen, such as DNA, in a sample solution to be examined, hybridizes with biological material, such as DNA probes.

The gel retaining layer 5 preferably has a constitution that can be readily detached from the electrophoresis portion 100 that has been assembled. That is by disposing a spacer having a gel retaining layer receiving portion at the position of the gel retaining layer 5 in FIG. 3, a structure is achieved in which the gel retaining layer can be disposed in the gel retaining layer receiving portion via an insertion method.

It is sufficient that the area of the sample solution storage portions 4 and 6 joined to the gel retaining layer 5 is equal to or greater than the cross-sectional area of the gel retaining portion of the gel retaining layer 5.

The shape of the sample solution storage portions 4 and 6 can be square, or various other shapes such as a circle or polygon. The material of the sample solution spacers 11 and 12 is preferably one that does not cause leakage of the sample solution and the washing solution, is chemically stable, hardly leaches ions and other components into the sample solution and washing solution, and adsorbs a minimal quantity of specimen molecules in the sample solution. Example materials include butyl rubber, nitrile rubber, silicone rubber, Teflon (registered trademark) resin, acrylic resin, and polycarbonate resin.

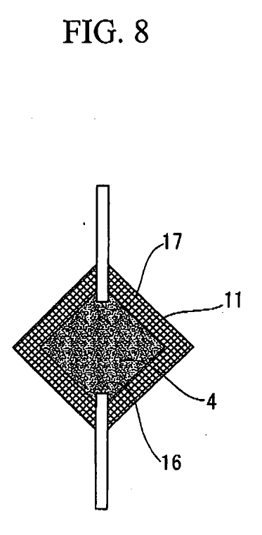

In the present example, as shown in FIG. 3 and FIG. 8, a sample solution inlet/outlet 16 that feeds and drains the sample solution from the lowermost part of the sample solution storage portion 4, and a sample solution storage portion air supply/exhaust port 17 that supplies air from the uppermost portion of the sample solution storage portion 4 to the space in the sample solution storage portion 4 and exhausts air therefrom are formed in the sample solution spacer 11. Similarly, a sample solution inlet/outlet 18 that feeds and drains the sample solution from the lowermost part of the sample solution storage portion 6, and a sample solution storage portion air supply/exhaust port 19 that supplies air from the uppermost portion of the sample solution storage portion 6 to the space in the sample solution storage portion 6 and exhausts air therefrom are formed in the sample solution spacer 12. The position of the air supply/exhaust ports is not limited to the uppermost portion, and the positions of the sample solution inlet/outlets are not limited to the lowermost portion.

When feeding the sample solution into the sample solution storage portions, a method of aspiring gas in the sample solution storage portions or a method of forcing the sample solution into the sample solution storage portions is adopted. In either case the air supply/exhaust ports are used as air exhaust ports. In order to introduce their sample solution without remaining air bubbles in the sample solution storage portions, a device that feeds the sample solution from the lowermost portions of the sample solution storage portions and removes air from the uppermost portions is preferred here. Also, when draining the sample solution from the sample solution storage portions, a method of forcing gas into the sample solution storage portions or a method of suctioning the sample solution from the ample solution storage portions is adopted, In either case, the air supply/exhaust ports are used as air supply ports.

At least one of the sample solution inlet/outlets 16 and 18 and at least one of the air supply/exhaust ports 17 and 19 should be provided. Also, the material of the sample solution inlet/outlets 16 and 18 and the air supply/exhaust ports 17 and 19 is preferably one that does not cause leakage of the sample solution and the washing solution,is chemically stable, hardly leaches ions and other components into the sample solution and the washing solution, and adsorbs a minimal quantity of specimen molecules in the sample solution.

It is preferable that the volumetric capacities of the sample solution storage portions 4 and 6 be as small as possible. The smaller the volumetric capacity, the higher the specimen concentration in the sample solution, which can raise the detection sensitivity.

A solution that contains a specimen of a biological material can be used as the sample solution to be fed into the sample solution storage portions 4 and 6. For example, a solution that contains DNA, RNA, proteins, peptides, surfactants, carbohydrates, and the like can be used. The specimen of biological material can be labeled with a publicly known method using a fluorescent material, a radioisotope, or a chemical luminescent substance.

The semi-permeable membranes 3 and 7 pass small molecules such as water and salts in the solution without passing the specimen. For example, hydro gel, such as a gelatin film, an acetate film, an acrylamide polymer, and polyvinyl alcohol, and a regenerated cellulose film and the like can be used. Among these, an acetate film is preferable in consideration of its mechanical strength and ease of handling and availability.

By using the semi-permeable membranes 3 and 7, for example, the buffer solution of the buffer solution storage portions 2 and 8 and the sample solution of the sample solution storage portions 4 and 6 penetrate the semi-permeable membranes 3 and 7, to be exchanged. The biological material to be detected that is contained in the sample solution storage portions 4 and 6 does not pass through the semi-permeable membranes 3 and 7, and thus remains in the sample solution storage portions 4 and 6. Accordingly, the hybridization reaction can be efficiently performed by having the biological material to be detected efficiently brought into contact with the carrier gel of the gel retaining layer while being able to adjust the migration conditions, such as temperature of the sample solution, via the buffer solution.

It is still more preferable to select a semi-permeable membrane that has a suitable cut-off molecular weight in accordance with the biological material that is the detection target. The cutoff molecular weight is the minimum molecular weight that retains 90% after 17 hours of dialysis. Semi-permeable membranes are available on the market with a cut-off molecular weight of several thousand to several hundreds of thousands. In other words, it is preferable to use a semi-permeable membrane having a cut-off molecular weight smaller than the molecular weight of the biological material to be detected.

In the present example, buffer solution feed ports 14 and 20 which feed the buffer solution from the lowermost portion of the buffer solution storage portions 2 and 8, respectively, are formed in the buffer solution spacers 10 and 13, respectively. Buffer solution drain ports 15 and 21 which drain the buffer solution from the uppermost portion of the buffer solution storage portions are formed, respectively, in the buffer solution spacers 10 and 13. The position of the buffer solution feed ports is not limited to the lowermost portion, and the position of the buffer solution drain ports is not limited to the uppermost portion.

The material used for the buffer solution spacers 10 and 13 may be one that does not cause a leakage of the buffer solution and is chemically stable with minimal leaching of ions and other components into the buffer solution. Example materials include butyl rubber, nitrile rubber, silicone rubber, Teflon (registered trademark) resin, acrylic resin, and polycarbonate resin. The shape of the buffer solution storage portions 2 and 8 is not particularly limited, with shapes such as a square, circle, or polygon possible

There is no limitation on the number of buffer solution feed ports 14 and 20 and the number of buffer solution drain ports 15 and 21. The material used for the buffer solution feed ports 14 and 20 and the buffer solution drain ports 15 and 21 is preferably one that does not cause a leakage of the buffer solution and is chemically stable with minimal leaching of ions and other components into the buffer solution.

Electrolytic solutions, such as a tris-boric acid buffer (TB), a tris-acetic acid buffer (TA), and sodium chloride-sodium citrate (SSC), can be used as the buffer solution that is held in the buffer solution storage portions 2 and 8.

Although the material constituting the electrodes 1 and 9 is not particularly limited, it is suffice to be a conductive material. Excluding the case of using reversible electrodes, it is suitable to use a material that is chemically stable with minimal leaching of ions and other components into the buffer solution, with platinum being preferable, for example. The electrodes 1 and 9 may be flat shaped or be formed in various other shaper. Also, the electrodes 1 and 9 can be constituted from a plurality of electrodes.

The spatial relationship of the electrodes 1 and 9 and the buffer solution storage portions 2 and 8 is preferably of a construction such that the electrodes 1 and 9 are in contact with at least the buffer solution storage portions 2 and 8, respectively, and gas generated from the electrodes 1 and 9 as a result of electrolysis is able to be drained from the buffer solution storage portions 2 and 8 together with the buffer solution. A structure is also possible in which the electrodes 1 and 9 are immersed in the buffer solution storage portions 2 and 8, respectively.

Moreover, in FIG. 3, some or all of the buffer solution feed ports 14 and 20 and the buffer solution drain ports 15 and 21 may also function as electrodes. In such a case, the electrodes 1 and 9 become unnecessary.

The electrodes 1 and 9, the buffer solution spacers 10 and 13, the send-permeable membranes 3 and 7, and the sample solution spacers 11 and 12 need not be constituted as independent components. For example, at least two among the electrode 1, the buffer solution spacer 10, the semi-permeable membrane 3, and the sample solution spacer 11 can be used as a component manufactured as one piece. Moreover, they can all be used as one piece. In addition, a configuration is also possible that forms an electrophoresis portion by pressure contacting, adhesion, or joining of these members and components.

In the present example, the electrophoresis portion 100 is configured so that the stacking planes of the electrode 1, the buffer solution storage portion 2, the semi-permeable membrane 3, the sample solution storage portion 4, the gel retaning layer 5, the sample solution storage portion 6, the semi-permeable membrane 7, the buffer solution storage portion 8 and the electrode 9 are positioned in a vertical direction. However, the stacking planes of these component members can be positioned in a horizontal direction, vertical direction or any other direction. However, they are preferably positioned in the vertical direction in order to efficiently discharge gas from the sample solution storage portions 4 and 6 and the buffer solution storage portions 2 and 8.

In the present example, a supply mechanism that feeds the sample solution or the washing solution to the sample solution storage portions 4 and 6 or drains it therefrom is connected (hereinafter referred to as a “sample solution supply mechanism”). As shown in FIG. 1, the sample solution supply mechanism comprises a sample solution container 32 that stores the sample solution, a sample solution feed/drain pump 37 for feeding or draining the sample solution from the sample solution container 32 to or from the sample solution storage portions 4 and 6, piping therebetween, an air supply/exhaust port 55, and a first flow passage switching device 35. The “flow passage switching device” refers to a valve mechanism or to a fluid control mechanism in which flexible piping is directly pressed onto one or more connection ports to form a pressure connection, or a connection means such as a coupler is provided on the piping and connection ports, to switch flow paths by attachment or detachment of the connection ports and piping.

The sample solution container 32 shown in FIG. 1 is connected by piping to the sample solution inlet/outlets 16 and 18 shown in FIG. 2 and FIG. 3, and the sample solution feed/drain pump 37 is connected by piping to air supply/exhaust ports 17 and 19 for the sample solution storage portion shown in FIG. 2 and FIG. 3.

The sample solution supply mechanism moreover has a washing solution server 38 that stores the washing solution prepared in advance; a washing solution feed/drain pump 40 that feeds the washing solution from the washing solution server 38 to the sample solution storage portions 4 and 6 or drains it therefrom; a second flow passage switching device 33 that switches the type of solution fed/drained to/from the sample solution storage portions 4 and 6 to the sample solution or the washing solution; a drained washing solution container 41 that collects the washing solution drained from the sample solution storage portions 4 and 6; a third flow passage switching device 39 that switches the washing solution flow path to feeding from the washing solution sever 38 to the sample solution storage portions 4 and 6 or draining from the sample solution storage portions 4 and 6 to the drained washing solution container 41; and furthermore has a fourth flow passage switching device 36 that switches the operating pump to the sample solution feed/drain pump 37 or the washing solution feed/drain pump 40.

As the washing solution, a tris-boric acid buffer (TB), a tris-acetic acid buffer (TA), and sodium chloride-sodium citrate (SSC), and the like can be used.

When the sample solution supply mechanism of the present example opens the flow path to the sample solution container 32 side using the second flow passage switching device 33, the sample solution stored in the sample solution container32 is fed to the sample solution storage portions 4 and 6 through the sample solution inlet outlets 16 and 18 by means of suction applied by the sample solution feed/drain pump 37. The amount of the sample solution that is fed ran be monitored by a fluid detection sensor 34.

After termination of the electrophoresis, the sample solution is drained. First, the air supply/exhaust port 55 and the ample solution feed/drain pump 37 are communicated to each other using the first flow passage switching device 35 so that air is suctioned to the sample solution feed/drain pump 37. Next, the sample solution feed/drain pump 37 and the sample solution storage portions 4 and 6 are communicated to each other using the first flow passage switching device 35, In this sate the sample solution feed/drain pump 37 is set to the push-out side, and air is fed to the sample solution storage portions 4 and 6 to drain the sample solution held in the sample solution storage portions 4 and 6.

Next, the washing solution is fed to the sample solution storage portions 4 and 6. Using the second flow passage switching device 33 and the third flow passage switching device 39, the washing solution server 38 and the sample solution storage portions 4 and 6 are made to communicate with each other. In this state, the washing solution feed/drain pump 40 is set to the suction side, and the washing solution stored in the washing solution server 38 fed from the sample solution inlet/outlets 16 and 18 to the sample solution storage portions 4 and 6. The amount of the washing solution that is fed can be monitored by the fluid detection sensor 34.

The draining of the washing solution is performed according to the following procedure. First, using the fourth flow passage switching device 36 and the first flow passage switching device 35, the air supply/exhaust port 55 and the washing solution feed/drain pump 40 are communicated to each other so that air is suctioned to the washing solution feed/drain pump 40. Next, the third flow passage switching device 39 is set to the side to drain to the drained washing solution container 41. Furthermore, the washing solution feed/drain pump 40 and the sample solution storage portions 4 and 6 arc communicated to each other using the first flow passage switching device 3. In this state, the washing solution feed/drain pump 40 is set to the push-out side, and air is fed to the sample solution storage portions 4 and 6 to drain the washing solution held in the sample solution storage portions 4 and 6.

The feeding operation and drain operation for the washing solution can be repeated a plurality of times. Also, where the washing solution is held in the sample solution storage portions 4 and 6, it can also be removed by applying a voltage across the electrodes to cause electrophoresis of unbound specimens.

When contamination due to the sample solution containing a radioisotope, mutagen, and the like poses a problem in the sample solution feeding mechanism, it is preferable to use a fluid control mechanism in which flexible piping that readily enables discarding of the contaminated portion is directly pressed onto one or more connection ports to form a pressure connection, or a connection means such as a coupler is provided on the piping and connection ports, to switch flow paths by attachment or detachment of the connection ports and piping.

While both the sample solution feed/drain pump 37 and the washing solution feed/drain pump 40 are provided in the present example, a structure is also possible in which the sample solution or the washing solution is fed to the sample solution storage portions 4 and 6 or drained therefrom by a single pump. Moreover, a constitution is possible that further adds a flow path switching device to enable selective use of a plurality of sample solutions and washing solutions.

In the present example, the sample solution storage portions 4 and 6 share the sample solution feeding mechanism. However, solution feeding mechanisms can also be independently installed for the sample solution storage portions 4 and 6 for feeding and draining of the sample solution or washing solution,

Also, it is preferable to provide a sample solution supply mechanism that selectively feeds sample solutions or washing solutions of which one or more of a concentration, a temperature, and a composition differ to the sample solution storage portions 4 and 6.

For example, a second washing solution server is installed that holds a washing solution of which one or more of the concentration, temperature and composition differs from the washing solution stored in the washing solution server 38. Then, by means of the flow path switching device, which switches to one of the second washing solution server and the washing solution server 38, and the washing solution feed/drain pump 40, the washing solution can be maintained in the optimum conditions.

In the present example, a supply mechanism that feeds the buffer solution to the buffer solution storage portions 2 and 8 and drains it therefrom (hereinafter referred to as the “buffer solution supply mechanism”) is provided. As shown in FIG. 1, the buffer solution supply mechanism is constituted from buffer solution servers 23 and 27 that store the buffer solution prepared in advance; buffer solution sending pumps 24 and 28 that send the buffer solution from the buffer solution servers 23 and 27 to the buffer solution storage portions 2 and 8; piping therebetween; and buffer solution flow path switching device 26 and 30 that switches to draining the buffer solution drained from the buffer solution storage portions 2 and 8 to a drained buffer solution container 31 or to recycling it between the buffer solution servers 23 and 27. The buffer solution servers 23 and 27 are connected to the buffer solution feed ports 14 and 20. Piping is connected to the buffer solution drain ports 15 and 21 shown in FIG. 3, and this piping extends to the drained buffer solution container 31 and the buffer solution servers 23 and 27.

When the buffer solution flow path switching device 26 and 30 is set to he opened to the drained buffer solution container 31 in the buffer solution supply mechanism of the present example, the buffer solution is sent from the buffer solution servers 23 and 27 through the buffer solution feed ports 14 and 20 to the buffer solution storage portions 2 and 8 by the buffer solution sending pumps 24 and 28, from where it is subsequently drained to the drained buffer solution container 31.

When the buffer solution flow path switching device 26 and 30 is set to circulate the buffer solution between the buffer solution storage portions 2 and 8 and the buffer solution servers 23 and 27, the buffer solution can circulate between the buffer solution server 23 and the buffer solution storage portion 2, and between the buffer solution server 27 and the buffer solution storage portion 8.

By thus continuously or intermittently running the buffer solution sending pumps 24 and 28 for suction or drain in the buffer solution supply mechanism, the buffer solution can be continuously or intermittently fed to the buffer solution storage portions 2 and 8 or drained therefrom. In addition, suction pumps can be installed on the opposite sides of the buffer solution sending pumps 24 and 28 for the buffer solution storage portions 2 and 8 in the buffer solution supply mechanism.

In FIG. 1, the buffer solution supply mechanism, that supplies the buffer solution to the buffer solution storage portions 2 and 8, is independently installed for each buffer solution storage portion. However, a portion or all thereof may be installed in a shared manner. Furthermore, a buffer solution supply mechanism can also be provided that comprises a plurality of buffer solution servers of which each stores a buffer solution of which one or more of a concentration, a temperature, and a composition differs, a buffer solution sending pump, and a flow path switching device that switches the buffer solution to be fed to the buffer solution storage portions 2 and 8 to a buffer solution server. Thereby, buffer solutions of which one or more of a concentration, a temperature, and a composition differ can be selectively supplied to the buffer solution storage portions 2 and 8. By doing so, the concentration, temperature, and composition of the buffer solution can be maintained at the optimal conditions for hybridization and cleaning.

A suitable method of use in the first embodiment of the present invention will be described below with reference to FIG. 1 and FIG. 2.

A solution of a biological material such as DNA is prepared and labeled by a publicly known method to make the sample solution. The sample solution, buffer solution and washing solution prepared in advance are stored in the sample solution container 32, the buffer solution servers 23 and 27, and the washing solution server 38, respectively. First, by setting the second flow passage switching device 33 to the sample solution side and the sample solution feed/drain pump 37 to the suction side, the sample solution is fed from the sample solution container 32 to the sample solution storage portions 4 and 6.

Also, by setting the buffer solution flow path switching device 26 and 30 to circulate between the buffer solution storage portions 2 and 8 and the buffer solution servers 23 and 27, respectively, and running the buffer solution sending pumps 24 and 28, the buffer solution is circulated therebetween.

Next, by applying a voltage across the electrodes 1 and 9, with the electrode 1 serving as the positive electrode and the electrode 9 serving as the negative electrode, the DNA specimen contained in the sample solution is made to migrate. For example, a negatively charged DNA specimen will migrate from the sample solution storage portion 6 into a carrier gel 22 of the gel retaining layer 5. The DNA specimen specific to a DNA probe immobilized in the carrier gel 22 hybridizes with the DNA probe to he retained in the carrier gel 22. A DNA specimen that is not complimentary to the DNA probe will migrate through the sample solution storage portion 4 in the direction of the semi-permeable membrane 7 without hybridizing with the DNA probe. A DNA specimen that does not hybridize with a DNA probe will be referred to as an “unbound specimen”.

Here, if the polarity of the electrodes is reversed so that the electrode 1 is an anode and the electrode 9 is a cathode, an unbound specimen will re-migrate from the sample solution storage portion 4 into the carrier gel 22. Accordingly, by reversing the voltage applied on the electrodes, an unbound specimen can be made to travel both ways between the sample solution storage portion 4 and the sample solution storage portion 6, which can increase the hybridization efficiency with the DNA probe.

By continuing to run the buffer solution sending pumps 24 and 28 while the voltage is being applied, at the same time as feeding the buffer solution from the buffer solution feed ports 14 and 20 to the buffer solution storage portions 2 and 8, restively, gas that is generated on the surfaces of the electrodes 1 and 9 is drained from the buffer solution drain ports 15 and 21 together with the buffer solution.

After terminating the voltage application, the fourth flow passage switching device 36 is set to the washing solution feed/drain pump 40 side, the third flow passage switching device 39 is set to the feed side, and the washing solution feed/drain pump 40 is set to the suction side to suction the washing solution from the washing solution server 38. Thereby, the sample solution in the sample solution storage portions 4 and 6 is replaced by the washing solution. By subsequently applying a voltage between the electrode 1 and the electrode 9, only the negatively charged unbound specimens migrate to the sample solution storage portion on the positive electrode side to be removed from the gel retaining layer 5. Here, the removal of said unbound specimens can also be performed while agitating the washing solution of the sample solution storage portions 4 and 6 by suctioning or draining the washing solution feed/drain pump 40.

After terminating the voltage application, by setting the third flow passage switching device 39 is set to the draining side and setting the washing solution feed/drain pump 40 to the push-out side, the washing solution containing the unbound specimens is drained from the sample solution storage portions 4 and 6 to the drained washing solution container 41.

Next, the gel retaning layer 5 is removed, and the biological material retained in the carrier gel 22 is detected using a detection method compatible with the labeling method used for the sample solution.

In the present embodiment, it is preferable that the buffer solution feedports 14 and 20 are formed at the lowermost portion of the buffer solution spacers 10 and 13, respectively, and the buffer solution drain ports 15 and 21 are formed at the uppermost portion of the buffer solution ports 10 and 13, respectively. This enables the gas generated near the electrodes 1 and 9 when voltage is applied hereto to efficiently discharge from the buffer solution storage portions 2 and 8. According to this structure, the moving rate of the biological material in the sample solution storage portions 4 and 6 and the diffusion rate in the carrier gel 22 are improved, and electrophoresis can be performed without causing problems such as the formation of an insulating layer causing by gas generated from the electrodes 1 and 9, and thus detection accuracy improves.

Also, since the buffer solution can be continuously supplied to the buffer solution storage portions 2 and 8 using the buffer solution supply mechanism, the ion concentration can be made uniform in the buffer solution storage portions 2 and 8 and the sample solution storage portions 4 and 6 that make contact therewith via the semi-permeable membranes, and thus the detection accuracy can be increased.

In the present example, a temperature control mechanism is provided for heating or cooling the buffer solution supplied to the buffer solution storage portions 2 and 8.

As shown in FIG. 1, the temperate control mechanism comprises heat exchangers 25 and 29 that heat or cool the buffer solution supplied to the buffer, solution storage portions 2 and 8 to a specified temperature, and a temperature controller 42.

Heating or cooling the buffer solution by operating the heat exchangers 25 and 29 in accordance with a signal set in advance in the temperature controller 42 in such a temperature control mechanism can maintain the buffer solution storage portions 2 and 8 at the specified temperature, and thus the temperature of the sample solution in the sample solution storage portions 4 and 6 and the temperature of the carrier gel 22 in the gel retaining layer 5 can be held constant.

The method of heating and cooling the buffer solution is not particularly limited For example, a heat-medium-circulation method that circulates a heat medium between the heat exchangers 25 and 29 and the temperature controller 42, and a method that performs heating and cooling using a Peltier device as the heat exchangers 25 and 29 can be used.

Furthermore, a mechanism can be provided in which a temperature detecting element, such as thermocouple, is installed inside or near the exterior of the sample solution storage portions 4 and 6, with the temperature of the buffer solution being optimally adjusted in accordance with a signal from the temperature detecting element.

In the present invention, the electrode 1 and the electrode 9 are connected via electrical wiring and a signal generator mechanism. The signal generator mechanism comprises an arbitrary waveform generator 43 that sets or selects an arbitrary waveform by an external signal, sets the output voltage and can control the ON/OFF of the output; and a coordination control device 44 that can send a control signal to the arbitrary waveform generator 43 according to a sequence and a time set beforehand. Using this signal generator mechanism, an arbitrary voltage having an arbiter waveform: containing a direct current can be applied on the electrophoresis portion in accordance with the predetermined sequence and time.

In the present example, the arbitrary waveform generator 43, the temperature controller 42, buffer solution sending pumps 24 and 28, fluid detection sensor 34 that monitors the feed rate and temperature, and the like of the sample solution or the washing solution, the buffer solution flow path switching device 26, the second flow passage switching device 33, the first flow passage switching device 35, and the third flow passage switching device 39 are connected by signal circuitry to the coordination control device 44 to thereby form a coordination control mechanism.

In such a coordination control mechanism, feed rate or temperature signals of the sample solution or the washing solution detected by the fluid detection sensor 34 are sent to the coordination control device 44. Signals for controlling the buffer solution sending pumps 24 and 28, the temperature controller 42, and the arbitrary waveform generator 43 are then sent from the coordination control device 44 to the buffer solution sending pumps 24 and 28, the temperature controller 42, and the arbitrary waveform generator 43.

Using such a coordination control mechanism can corporate the buffer solution supply mechanism and the sample solution supply mechanism, or can coordinate the buffer solution supply mechanism, the sample solution supply mechanism and the signal generator mechanism. Accordingly, electrophoresis can be continued with the temperature, composition, and concentration of the buffer solution and the sample solution as well as the current that is applied on the gel retaining layer 5 maintained at optimum conditions.

Moreover, the operation of the flow path switching device and the operation of the temperature control mechanism can be carried out in coordination.

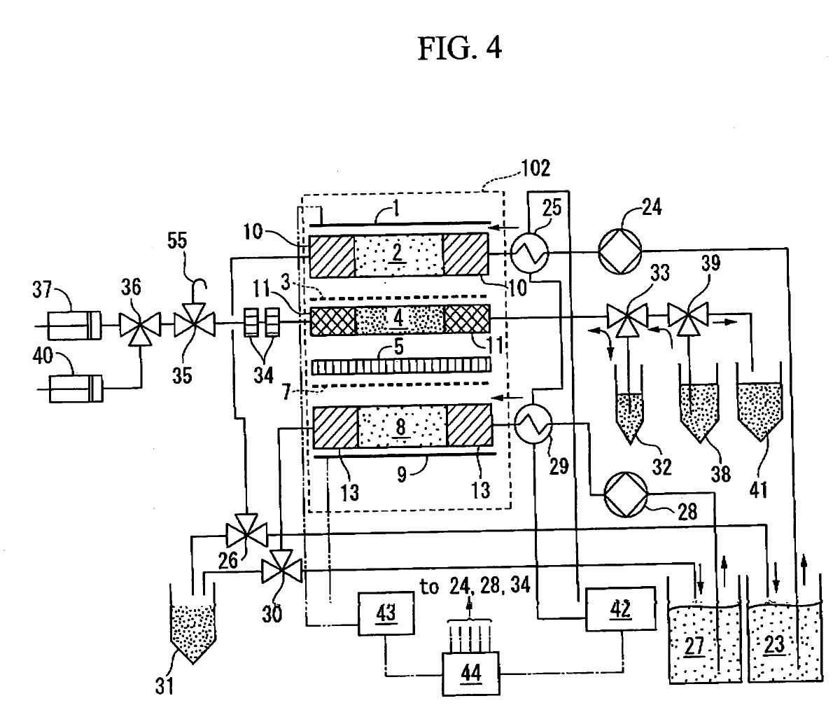

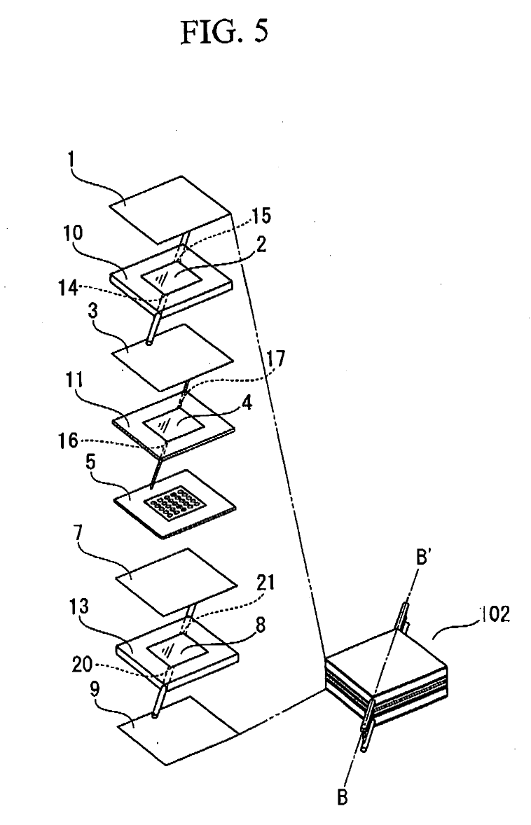

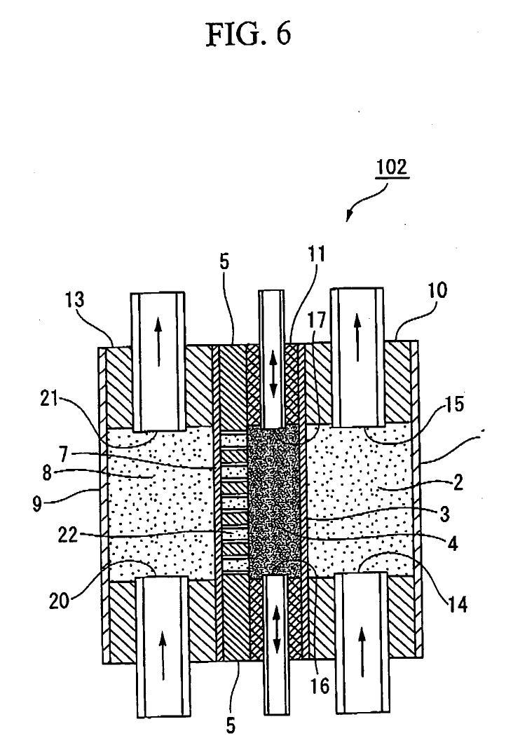

FIGS. 4 to 6 show the electrophoresis apparatus according to a second embodiment of the present invention, having a structure in which the sample solution storage portion 4 is disposed on only one side of the gel retaining layer.

In the present example, the sample solution is fed to the sample solution storage portion 4 from the sample solution container 32 similarly to the first embodiment of the present invention. When a voltage is applied across the electrodes 1 and 9, with the electrode 1 serving as the anode and the electrode 9 serving as the cathode, a negatively charged specimen contained in the sample solution storage portion 4 will migrate from the sample solution storage portion 4 into the carrier gel 22 of the gel retaining layer 5 by the applied voltage. A DNA specimen that is complimentary to a DNA probe held in the carrier gel 22 hybridizes with the DNA probe to be retained in the carrier get 22. A specimen that is not complimentary to the DNA probe will pass through the gel retaining layer 5 to be blocked by the semi-permeable film 7, and thus condenses between the send-permeable film 7 and the gel retaining layer 5.

Here, when the voltage that is applied on the electrodes is reversed so that the electrode 1 is the cathode and the electrode 9 is the anode, a negatively charged unbound specimen will migrate to the sample solution storage portion 4. The unbound specimen in the sample solution storage portion 4 is then drained to the drained washing solution container 41.

### EXAMPLES

### Example 1

(Manufacturing a DNA Chip for Gel Retaining Layer)

Two porous plates were prepared, each being an SUS304 plate measg 35 mm long, 35 mm wide, and 0.1 mm thick having 25 holes with a diameter of 0.32 mm formed in the center region measuring 2.1 mm×2.1 mm of each plate, the holes being formed in five columns and five rows at intervals of 0.42 mm. These porous plates were stacked, with polycarbonate hollow fibers (having an outside diameter of 0.28 mm, an inner diameter of 0.16 mm, and a length of 100 cm) passed through each of the holes. Next, the two porous plates were separated so that one is 10 cm and the other is 60 cm from one end of the fibers, with a 50-cm gap thereby being formed between the porous plates. These porous plates and the hollow fibers therebetween were then enclosed in a 10 mm-thick polytetrafluoroethylene plate.

Next, this enclosure was filled with a polyurethane resin (Nipporan 4276, Collonate 4403 manufactured by Japanese Polyurethane Industry Co.) colored by carbon black (MA1000 manufactured by Mitsubishi Chemicals Corp.). The resin was then cured by being left to stand for one week at room temperature. Afterward, the porous plates and the polytetrafluoroethylene plate enclosure were removed to obtain a hollow-fiber arranged body of a rectangular shape measuring 20 mm×20 mm and 50 mm long. Twenty-five hollow fibers were arranged in the center portion measure 2.1 mm×2.1 mm of the cross section of the hollow-fiber arranged body.

A mixed solution was then prepared containing 4.5 parts by weight of N, N-dimethylacrylamide, 0.5 part by weight of N,N-methylenebisacrylamide, 0.1 part by weight of 2, 2′-azobis(2-aminopropane) dihydrochloride, and 95 parts by weight of water. This mixed solution was fed into the hollow portions of 22 hollow fibers of the hollow-fiber arranged body. Into the remaining three hollow fibers was fed a solution containing 40-base DNA added to said mixed solution at a concentration of 5 mmol/ml. Polymerization was then conducted for three hours at 70° C. to generate a gel material in the hollow portions of the hollow fibers. Afterward it was cut into slices 0.5 nm thick, which were further trimmed around the edges to obtain DNA chips each measuring 12 mm×12 nm×0.5 mm.

Next, the electrophoresis apparatus shown in FIGS. 1 to 3 was assembled using the following components. That is, the aforementioned DNA chip was used as the gel retaining layer 5, and a semi-permeable membrane comprising Spectopore (with a molecular mass cutoff of 3500) manufactured by Spectrum Medical Industries was used as the semi-permeable membranes 3 and 7. Also, butyl-rubber buffer solution spacers 10 and 13, butyl-rubber sample solution spacers 11 and 12 and platinum electrodes 1 and 9 were arranged in the constitution shown in FIG. 1 and pressed together to form the electophoresis portion 102. Also, the arbitrary waveform generator 43 was connected to the electrodes 1 and 9, and the heat exchangers 25 and 29 and the temperature controller 42 were installed.

The buffer solution storage portion was one measuring 8 mm long, 8 mm wide and 2 mm thick, having a volumetric capacity of 128 microliters, while the sample solution storage portion was one measuring 8 mm long, 8 mm wide and 1 mm thick, having a volumetric capacity of 64 microliters.

First, 50 ml of a 0.5×TB−15 mM NaCl solution was poured into each buffer solution server 23 and 27, the buffer solution flow path switching device 26 and 30 were set to the circulation side, and the buffer solution sending pumps 24 and 28 were activated to circulate the buffer solution between the buffer solution server 23 and the buffer solution storage portion 2 and between the buffer solution server 27 and the buffer solution storage portion 8, respectively. At this time, the buffer solution was fed from the lowermost portion of the buffer solution storage portions 2 and 8 through the buffer solution feed ports 14 and 20, and the buffer solution was drained from the uppermost portion of the buffer solution storage portions 2 and 8 through the buffer solution drain ports 15 and 21. The temperature of the buffer solution was set to 45° C. using the temperature controller 42.

The sample solution was made by dissolving 100 fmol of a 40-base pair DNA specimen a, labeled with Cy™5 dye (excjuition wavelength 635 nm, detection wavelength 660 nm), that can complimentarily bind to DNA that is immobilized with said base material gel in the DNA chip (hereinafter referred to as “probe A”), and 100 fmol of a 40-base pair DNA specimen b, labeled with Cy™3 dye (excitation wavelength 580 nm, detection wavelength 570 nm), that does not complimentarily bind to the probe A in 100 microliters of the 0.5×TB−15 mM NaCl solution. After storing the sample solution in the sample solution container 32, the fourth flow passage switching device 36 is set to the sample solution side and the sample solution feed/drain pump 37 is set to the suction side, whereby the sample solution is suctioned and fed to the sample solution storage portions 4 and 6.

The washing solution comprises an 0.5×TB−15 mM NaCl solution that is stored in the washing solution server 38.

A direct current is applied by the arbitrary waveform generator 43 so that the electrode 1 is the positive electrode and the electrode 9 is the negative electrode thereby causing the migration of the DNA specimen a and the DNA specimens a and b contained in the sample solution. The applied voltage is 3V, and the application time is 10 minutes.

Thereafter, the sample solution feed/drain pump 37 was set to the push out side, and the sample solution was drained from the sample solution storage portions 4 and 6 to the drained washing solution container 41. Without a break, the fourth flow passage switching device 36 was set to the washing solution side and the third flow passage switching device 39 was set to the feed side, whereby the washing solution was suctioned from the washing solution server 38 using the washing solution feed/drain pump 40. By continuously replacing the sample solution in the sample solution storage potions 4 and 6 with the cleaning solution, the unbound specimens were flushed out of the sample solution storage portions 4 and 6.

Next, the direct current voltage was applied with the polarity reversed using the arbitrary waveform generator 43, so that the electrode 1 was the negative electrode and the electrode 9 was the positive electrode. This removed the DNA specimens from the gel retaning layer 5 that had not bound with the probe A. The applied voltages this time was 3V, and the application time is 10 minutes.

After terminating the voltage application, the gel retaining layer 5 was removed and inspected under a fluorescence microscope with excitation wavelengths of 532 nm and 633 nm.

As a result, among the 25 carrier gel portions in the gel retaining layer 5, fluorescence emitted by the Cy™5 dye was detected only at the three locations where the probe A is immobilized, while fluorescence emitted by the Cy™3 dye was not detected at any of the locations.

### Comparative Example 1

Instead of concentrating the DNA specimen and hybridizing with the probe by electophoresis, hybridization and washing were performed similarly to Example 1, without applying a voltage while leaving to stand for 10 minutes, The gel retaining layer 5 comprising a DNA chip was then observed under a fluorescence microscope similarly to Example 1.

As a result, fluorescence emitted by the Cy™5 dye was detected only at the three locations where the probe A was immobilized, however, the fluorescence intensity was one-tenth or less compared with Example 1.

### Comparative Example 2

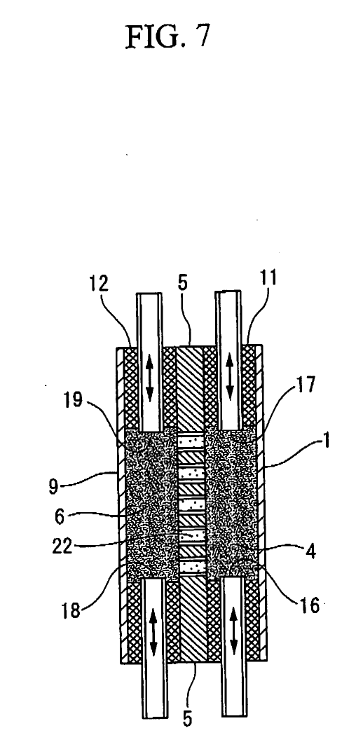

As shown in FIG. 7, hybridization and washing by electrophoresis were performed similarly to Example 1, except for using an electrophoresis apparatus that was not equipped with the buffer solution storage portions 2 and 8 and the semi-permeable membranes 3 and 7. The gel retaining layer 5 was observed under a fluorescence microscope similarly to Example 1.

As a result, neither the fluorescence emitted by the Cy™5 dye nor the fluorescence emitted by the Cy™3 dye were detected at any of the 25 carrier gel portions in the gel retaining layer 5. It is considered that the hybridization efficiency decreased due to decomposition of the nucleic acid specimens from the electrode reaction caused by electrolysis.

1. An electophoresis apparatus comprising:
a gel retaining layer;
one or two sample solution storage portions disposed on either side or both sides of said gel retaining layer;
two semi-permeable membranes disposed on the outer sides of said sample solution storage portions;
buffer solution storage portions disposed on the outer sides of said semi-permeable membranes;
a pair of electrodes disposed on the outer sides of said buffer solution storage portions; and
at least one liquid inlet/outlet respectively provided in each of the sample solution storage portions and the buffer solution storage portions.

2. An electrophoresis apparatus comprising:
a gel retaining layer;
one or two sample solution storage portions disposed on either side or both sides of said gel retaining layer;
two semi-permeable membranes disposed on the outer sides of said sample solution storage portions;
buffer solution storage portions disposed on the outer sides of said semi-permeable membranes; and
at least one liquid inlet/outlet respectively provided in each of the sample solution storage portions and the buffer solution storage portions, the liquid inlet/outlets of the buffer solution storage portions also functioning as electrodes.

3. The electrophoresis apparatus according to claim 1 or 2, wherein a first supply mechanism is connected that feeds and/or drains the buffer solution to and/or from the buffer solution storage portions.

4. The electophoresis apparatus according to claim 1 or 2, wherein a second supply mechanism is connected that feeds and/or drains the sample solution or the washing solution to and/or from the sample solution storage portions.

5. The electrophoresis apparatus according to claim 4, wherein a inlet/outlet that feeds or drains the sample solution or the washing solution is formed at the lowermost portion of the sample solution storage portions, and an air supply/exhaust port that feeds or drains the sample solution or the washing solution is formed at the uppermost portion of the sample solution storage portions.

6. The electrophoresis apparatus according to claim 1 or 2, wherein a feed port that feeds the buffer solution is formed at the lowermost portion of the sample solution storage portions, and a drain port that drains the buffer solution is formed at the uppermost portion of the sample solution storage portions.

7. The electrophoresis apparatus according to claim 1 or 2, wherein a temperature control mechanism is provided for heating or cooling to a specified temperature the buffer solution to be fed to the buffer solution storage portions.

8. The electrophoresis apparatus according to claim 1 or 2, wherein a buffer solution supply mechanism is provided for feeding buffer solutions that are different in terms of one or more of a concentration, a temperature, and a composition by switching between them to the buffer solution storage portions.

9. The electrophoresis apparatus according to claim 1 or 2, wherein a signal generator mechanism is provided for applying an arbitrary waveform and/or voltage on the electrodes according to a sequence and a time set beforehand.

10. The electophoresis apparatus according to claim 1 or 2, wherein a buffer solution supply mechanism that feeds buffer solutions that are different in terms of one or more of a concentration, a temperature, and a composition by switching between them to the buffer solution storage portions; a signal generator mechanism that applies an arbitrary waveform and/or voltage on the electrodes according to a sequence and a time set beforehand; and a coordination control mechanism that coordinates the operation of the buffer solution supply mechanism and the signal generator mechanism are provided.

11. The electrophoresis apparatus according to claim 10, wherein a sample solution supply mechanism that feeds buffer solutions or washing solutions that are different in terms of one or more of a concentration, a temperature, and a composition by switching between them to the sample solution storage portions; and a coordination control mechanism that coordinates the operation of the buffer solution supply mechanism, the signal generator mechanism, and the sample solution supply mechanism are provided.

12. The electrophoresis apparatus according to claim 1 or 2, wherein the gel retaining layer is a gel material held in one or more through-holes provided in a porous plate, a biological material being bound to this gel material.

13. The electrophoresis apparatus according to claim 12, wherein said biological material is DNA probes.

14. An electrophoresis method comprising the steps of:
feeding a sample solution or a washing solution to sample solution storage portions; and
applying a voltage across the pair of electrodes while feeding a buffer solution to a buffer solution storage portions using said electrophoresis apparatus, using the electrophoresis apparatus according to claim 1 or 2.

15. An electrophoresis method according to claim 14, wherein the sample solution or the washing solution is continuously or intermittently fed or drained to or from the sample solution storage portions.

16. An electrophoresis method according to claim 14, wherein the buffer solution is continuously or intermittently fed or drained to or from the buffer solution storage portions.

17. An electrophoresis method according to claim 14 comprising the steps of:
feeding the buffer solution from a liquid inlet/outlet at the lowermost portion of the buffer solution storage portions; and
draining the buffer solution from a liquid inlet/outlet at the uppermost portion of the buffer solution storage portions.
